(19) United States
(12) Patent Application Publication
(10) Pub. No.: US 2014/0276568 A1
(43) Pub. Date: Sep. 18, 2014

(54) SYSTEMS AND METHODS FOR DAMPENING FRICTION IN AN AUTOINJECTOR DEVICE

(71) Applicants: Morris Elijah Worden, New York, NY (US); Anthony Torris, Montclair, NJ (US); Alistair Bramley, Brooklyn, NY (US); Eric Freitag, Brooklyn, NY (US); Daniel Formosa, Piermont, NY (US)

(72) Inventors: Morris Elijah Worden, New York, NY (US); Anthony Torris, Montclair, NJ (US); Alistair Bramley, Brooklyn, NY (US); Eric Freitag, Brooklyn, NY (US); Daniel Formosa, Piermont, NY (US)

(21) Appl. No.: 13/836,606

(22) Filed: Mar. 15, 2013

(51) Int. Cl. G09B 23/28; A61M 5/32; A61M 5/315

(52) U.S. Cl. CPC G09B 23/285; A61M 5/31501; A61M 5/3157; A61M 5/3221
USPC 604/506; 604/220; 604/189; 434/262

(57) ABSTRACT

Systems, devices, and methods are described for using an autoinjector device. In one aspect, an autoinjector device comprises a housing and a plunger slidably mounted to the housing, the plunger including a tapered region. An actuator urges the plunger with respect to the housing from a storage position to a launch position, and a friction element about a portion of the tapered region resists a movement of the plunger from the storage position to the launch position.

### BACKGROUND

Individuals can self-administer medication using autoinjector devices. Autoinjectors are designed to be user-friendly because patients, unlike physicians or nurses, are not trained medical personnel. The autoinjector device is pressed against an injection site and automatically inserts a syringe needle into the injection site. After the medication has been delivered, the needle is refracted.

Despite the relative ease of use, an autoinjector device can still be intimidating for patients and particularly first-time users. It is important for a user to learn how to properly operate an autoinjector and to become comfortable with its use. Training and demonstration devices have been designed for a user to practice using an autoinjector. In order for a user to become adequately trained to use an autoinjector, the demonstration device should emulate a user's experience as realistically as possible. However, existing demonstration devices may not realistically emulate several aspects of the autoinjection process. There is therefore a need for training devices to realistically mimic the user's experience with an autoinjector.

### SUMMARY

Disclosed herein are systems, devices, and methods for using an autoinjector device. In one aspect, an autoinjector device comprises a housing and a plunger slidably mounted to the housing, the plunger including a tapered region. An actuator urges the plunger with respect to the housing from a storage position to a launch position, and a friction element about a portion of the tapered region resists a movement of the plunger from the storage position to the launch position.

In certain implementations, the tapered region has a variable diameter. In certain implementations, the tapered region has a central region between a proximal region and a distal region, and the proximal region is closer to the storage position and the distal region is closer to the launch position. In certain implementations, the proximal region has a diameter larger than a diameter of the central region. In certain implementations, the distal region has a diameter larger than a diameter of the central region. In certain implementations, the tapered region is tapered between the distal and central regions and between the proximal and central regions.

In certain implementations, the friction element presses against a wall of the housing when the plunger is urged from storage position to the launch position, resulting in a frictional resistive force. In certain implementations, the movement of the plunger from the storage position to the launch position causes a position of the friction element to be at a proximal region of the tapered region. In certain implementations, the plunger is configured to return to the storage position from the launch position.

In certain implementations, the tapered region includes a mechanism for reducing a resistive effect of the friction element when the plunger returns to the storage position from the launch position relative to the resistive effect of the friction element when the plunger moves from the storage position to the launch position. In certain implementations, the mechanism causes the friction element to bend when the plunger returns to the storage position from the launch position, wherein bending of the friction element results in a reduced amount of friction between the friction element and a wall of the housing. In certain implementations, the mechanism is a ridge on the distal region of the tapered region.

In certain implementations, the autoinjector device is a demonstration device used for simulating auto-injection. In certain implementations, the autoinjector device is configured to provide a first haptic feedback at an initiation of the plunger's movement from the storage position to the launch position. In certain implementations, a release of the actuator from at least one locking clip provides the first haptic feedback. In certain implementations, the autoinjector device comprises a puller coupled to the plunger and a cap, wherein the puller and the cap are configured to contact. The contact thereby provides the first haptic feedback.

In certain implementations, the autoinjector device is configured to provide a second haptic feedback at a conclusion of the plunger's movement from the storage position to the launch position. A release of the friction element causes the plunger to accelerate, and the second haptic feedback is provided when portions of the device contact. The portions may include a base and a puller coupled to the plunger, and the puller may be configured to contact an outer wall of a widened portion of the base.

In certain implementations, the autoinjector device is a medical device used for administering medication. In certain implementations, the plunger is a part of a syringe assembly including a needle and a fluid container.

In certain implementations, the friction element is a rubber ring.

In one aspect, a method for actuating an autoinjector comprises applying a first amount of resistance against a movement of a plunger slidably mounted to a housing from a storage position to a launch position. The method further comprises applying a second amount of resistance against a return of the plunger from the launch position to the storage position, wherein the second amount of resistance is smaller than the first amount of resistance.

In certain implementations, the resistance against the movement is caused by friction resulting from contact between a friction element about a portion of the plunger and a wall of the housing. In certain implementations, the method further comprises providing a first haptic feedback when the movement of the plunger from the storage position to the launch position initiates. In certain implementations, the method further comprises providing a second haptic feedback when the movement of the plunger from the storage position to the launch position concludes.

In one aspect, a system is disclosed for moving a plunger slidably mounted to a housing. The system comprises means for applying a first amount of resistance against a movement of the plunger from a storage position to a launch position and means for allowing the plunger to return to the storage position from the launch position. The system further comprises means for applying a second amount of resistance against the return of the plunger from the launch position to the storage position, wherein the second amount of resistance is smaller than the first amount of resistance.

In one aspect, a system is disclosed for moving a plunger slidably mounted to a housing. The system comprises a housing and a plunger slidably mounted to the housing, the plunger including a tapered region. Actuating means urges the plunger with respect to the housing from a storage position to a launch position, and resistive means about a portion of the tapered region resists a movement of the plunger from the storage position to the launch position.

Variations and modifications of these embodiments will occur to those of skill in the art after reviewing this disclosure. The foregoing features and aspects may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1A shows a cross sectional view of an illustrative autoinjector device in a storage position;

FIG. 1B shows a cross sectional view of the autoinjector device of FIG. 1A in a launch position;

FIG. 2 shows an exploded perspective view of components of the autoinjector device of FIGS. 1A and 1B;

FIG. 3A shows a cross sectional view of an illustrative autoinjector device in a storage position;

FIG. 3B shows a cross sectional view of the autoinjector device of FIG. 3A near a beginning of a transition from the storage position to a launch position;

FIG. 3C shows a cross sectional view of the autoinjector device of FIGS. 3A and 3B near a conclusion of the transition from the storage position to the launch position;

FIGS. 4A and 4B show two views of an illustrative friction element during a transition of an autoinjector device from a storage position to a launch position;

FIGS. 5A and 5B show two views of an illustrative friction element during a transition of an autoinjector device from a launch position to a storage position;

FIG. 6 shows an illustrative flow diagram for applying asymmetric bi-directional forces while an autoinjector device transitions between storage and launch positions; and

FIG. 7 shows an illustrative flow diagram for operating an autoinjector device.

### DETAILED DESCRIPTION

To provide an overall understanding of the systems, devices, and methods described herein, certain illustrative embodiments will be described. Although the embodiments and features described herein are specifically described for use in connection with autoinjector devices, it will be understood by one of ordinary skill in the art that the systems and methods described herein can be adapted and modified for other suitable applications and that such other additions and modifications will not depart from the scope hereof.

FIGS. 1A and 1B show cross sectional views of an autoinjector device 100, and FIG. 2 shows an exploded perspective view of components of the autoinjector device 100. The autoinjector device 100 includes a plunger 102 slidably mounted to a housing 101, which includes a base element 108. FIG. 1A shows the autoinjector device 100 in a “storage” position, where the autoinjector device 100 has yet to be discharged. In the storage position, the plunger 102 is in a position substantially adjacent to the top of a housing 101. In particular, in the storage position, a top of a notched region 104 of the plunger 102 is near the top of the base element 108. FIG. 1B shows the autoinjector device 100 in a “launch” position, from which the autoinjector device 100 is discharged. In the launch position, the plunger 102 is in a lower position substantially adjacent to the bottom of a housing 101. In particular, in the launch position, the tip 107 of the plunger 102 is below the bottom of the base element 108.

As the autoinjector device 100 transitions from a storage position as shown in FIG. 1A to a launch position as shown in FIG. 1B (e.g., during a discharge or injection of the autoinjector device 100), certain components of the autoinjector device 100 move with respect to other components that remain in a fixed position. In particular, the housing 101, including the base element 108, remains in a fixed position during the transition, while the plunger 102 and other components to which the plunger 102 is fixedly attached move with respect to the housing 101. For example, the plunger 102 is fixedly attached to a puller 112, which is fixedly attached to a piston 105 and an upper handle 110, and the puller 112, the piston 105, and the upper handle 110 all move with the plunger 102 during the transition from storage to launch positions.

The plunger 102 includes several regions, including the tip 107, a notched region 104, a body 113, and a tail 115. As depicted in FIGS. 1A and 1B, the body 113 has a diameter A, the notched region 104 has a variable diameter B, and the tip 107 has a variable diameter C. In addition, the diameter F of the tail 115 is relatively smaller than diameter A in order to fit within a portion of the puller 112 and the piston 105. A friction element 106 is positioned about a portion of the notched region 104. As shown in FIGS. 1A and 1B, the notched region 104 is a region of the plunger 102 with a variable or tapered diameter. In particular, one portion of the notched region 104 has a smaller diameter B than the body 113 (i.e., diameter A) and the tip 107 (i.e., diameter C). In an example, the friction element 106 may be a resistive element or a polymer ring surrounding the notched region 104. The inner diameter of the polymer ring may be sized appropriately such that the friction element 106 remains within the notched region 104. In particular, the inner diameter of the polymer ring may be greater than a diameter B of the notched region 104 but less than the diameters A and C. In addition, the outer diameter of the polymer ring may be near A or slightly larger than A. By using an appropriately sized friction element 106 that remains within the notched region 104, the elements of the autoinjector device 100 are configured to exert bi-directional resistive forces during injection and retraction. These bi-directional forces are explained in more detail in relation to FIGS. 4A, 4B, 5A, and 5B.

As shown in FIG. 2, the puller 112 is shaped like a hollow tube and is sized appropriately to surround a portion of the plunger 102. A tail portion 123 (shown in FIG. 2) of the puller 112 is sized to encompass the tail 115 of the plunger 102. As shown in FIGS. 1A and 1B, the plunger 102 is fixedly attached to the puller 112 at the tail portion 123 and the tail 115. In general, the plunger 102, the puller 112, and the piston 105 may be fixedly attached to each other at any of their other respective portions. The diameter of the hollow tube of the piston 105 is relatively larger than the diameter of the puller 112, which is relatively larger than the diameter of the body 113 of the plunger 102 (i.e., diameter A) to fit the plunger 102 therein. The piston 105 includes a lip 114, and the puller 112 includes a lip 119, the lips 114 and 119 positioned at an end opposite from the tail portion 123 of the puller 112. As shown in FIG. 2, the lip 119 is circular and continuously surrounds a circumference of the puller 112. The lip 114 of the piston 105 (not shown in FIG. 2) may also be circular, continuously surrounding a circumference of the piston 105. The lip 114 has a relatively wider outer diameter G than the remainder of the piston 105, and the lip 119 has a relatively wider outer diameter than the remainder of the puller 112. In some implementations, it is desirable to provide one or more haptic feedbacks signaling a beginning and/or an end of the injection. For example, at an end of the injection movement, the slightly widened diameter of the lip 114 enables the lip 114 to contact an element 121 of the housing 101, thereby providing a haptic feedback signaling the end of the injection. In addition, the haptic feedback may also or alternatively be provided with contact between the lip 119 and a ridge 116 of the base element 108. The ridge 116 is on the outer surface of a tube 117 of the base element 108. In general, the diameter of the lips 114 and 119 may be any size. In some implementations, the lip 119 and/or the lip 114 is not circular and is formed at discrete regions around a circumference of the puller 112 and/or the piston 105, respectively. A lip that is formed at discrete regions may still be configured to provide a haptic feedback when the lip comes in contact with another element. An implementation of providing one or more haptic feedbacks during the transition from the storage position to the launch position is described in detail in relation to FIGS. 3A, 3B, and 3C.

The base element 108 includes two hollow cylindrical portions: a tube 117 with an inner diameter D and a widened base portion 118 with an inner diameter E larger than D. The inner diameter D of the base element 108 may be sized appropriately to fit the widest diameter A of the plunger 102. That is, the inner diameter D of the base element 108 may be approximately or slightly larger than the diameter A of the body 113 so that the plunger 102 may fit within the inner wall 125 of the base element 108.

During a transition from the storage position (FIG. 1A) to the launch position (FIG. 1B), an actuator (i.e., the spring 220, a post, or any other suitable element for urging a movement of the plunger 102, not shown in FIG. 1A or 1B) urges the plunger 102 in a launch direction of the downward arrow 109 in FIG. 1A. As the plunger 102 moves in the launch direction, the friction element 106 (with an outer diameter relatively greater than the inner diameter D of the base element 108) presses against an inner wall 125 of the base element 108. The contact between the friction element 106 and the base element 108 produces a frictional force that resists the movement of the plunger towards the launch position. The frictional force urges the friction element 106 in a direction opposite of the arrow 109 towards proximal region 430 of the notched region 104, which is described further in FIGS. 4A and 4B. When the autoinjector device 100 is used for injection demonstration or training purposes, the resistive force emulates an effect that is caused by a drug's viscosity in a drug delivery device and therefore results in a more realistic user experience. In certain embodiments, the autoinjector device 100 may be a drug delivery device (e.g., the plunger 102 may include a syringe assembly and a needle), if it is desirable to provide a resistive force during injection. As an example, the drug may have a low viscosity, and it may be desirable to slow the injection process by applying a resistive force as described above. While it may generally be desirable to have a fast injection process, a possible advantage of slowing down the injection is to provide some pain relief compared to a faster injection.

As the autoinjector device 102 approaches the launch position, the frictional force resisting the injection movement is removed or reduced. In particular, in the launch position depicted in FIG. 1B, the body 113 of the plunger 102 is positioned within the tube 117 of the base element 108, and most of the notched region 104 is positioned within the widened base portion 118 of the base element 108. The hollow tube portion of the puller 112 is outside the base element 108, while the body 113 of the plunger 102 is within the base element 108. As the actuator urges the plunger 102 into the launch position, the notched region 104 of the plunger 102 approaches the widened base portion 118, and the widened inner wall of the base element 108 causes the friction element 106 to be released from being pressed against the inner wall of the tube 117. This results in a removal or reduction of the frictional force between the friction element 106 and the base element 108. In an example, gravity may cause the friction element 106 to drop to the bottom of the notched region 104, or towards the tip 107 of the plunger 102. This removes the frictional force resisting the injection movement. In another example, in the launch position, the friction element 106 may remain pressed against the inner wall of the base element 108 (at either the tube 117 or the widened base portion 118), against the outer wall of the notched region 104, or a combination thereof. In this case, the frictional force resisting the injection movement is reduced by relieving some of the force between the friction element 106 and the inner wall of the base element 108.

The autoinjector device 100 is resettable such that its components can return to the storage position from the launch position. In particular, during the return, the plunger 102 is urged in the direction 111 shown in FIG. 1B. In an example, the return from the launch position to the storage position may be facilitated by a recharging mechanism, including, for example, the recharging unit described in U.S. patent application Ser. No. ______ (Attorney Docket No. 106471-0012-102), filed concurrently herewith, and entitled “Systems and Methods for Recharging and Auto-Injector,” the disclosure of which is hereby incorporated by reference herein in its entirety. During the movement of the plunger 102 in direction 111, the friction element 106 remains near the tip 107 of the plunger 102, and this area of the notched region 104 may include a mechanism for reducing the effect of the friction element 106 that resists the movement of the plunger 102. An example of the mechanism included in the notched region 104 is described further in FIGS. 5A and 5B. A reduced resistive force while the plunger 102 moves in the direction 111, relative to the resistive force when the plunger 102 moves in the direction 109, may be desirable for conveniently resetting the autoinjector device 100 such that the device may be used multiple times. A reusable autoinjector device 100 may be desirable when the device is used for demonstration purposes. As an example, the autoinjector device 100 may be used to demonstrate a single-use drug delivery device. However, it is undesirable to have the demonstration device also be single-use, so the demonstration device may be resettable for multiple uses. For example, it may be desirable for the resetting of the autoinjector device 100 to occur quickly. In this case, reducing the amount of resistive force during the return of the plunger 102 to the storage position results in efficient resetting of the device. Furthermore, reducing the amount of resistive force enables the user to exert less force when the device is being recharged.

In another example, the reduced resistive force while the plunger 102 moves in the direction 111, relative to the resistive force while the plunger 102 moves in the direction 109, may be desirable for emulating the upward movement of a syringe in a drug delivery device. In particular, when the syringe returns to a storage position in a drug delivery device, there may be a reduced resistive force compared to during injection. The reduced resistive force results because the drug has been delivered, and the viscosity of the drug no longer exerts a resistive force. Therefore, reducing the resistive force during an upward movement of the plunger 102 may be desirable for realistically emulating a user's experience with a drug delivery device.

The autoinjector device 100 provides a more realistic user experience by producing asymmetric bi-directional forces during injection and recharging. Specifically, the autoinjector device 100 provides a first force that resists injection (e.g., during transition from the storage to launch position) and a second, reduced force when the device recharges (e.g., during return from the launch to storage position). In some embodiments, a user using the autoinjector device 100 can view on the outside of the autoinjector device 100 an indicator region 103, which may be colored differently than a remainder of the autoinjector device 100. The indicator region 103 may provide an indication of whether the autoinjector device 100 is in the storage or launch position, or any suitable indication of a progress of the injection process. For example, the height of the indicator region 103 may provide an indication to the user of a position of the autoinjector device 100.

In some embodiments, the friction element 106 is a ring that surrounds a portion of the notched region 104. The ring may be made of a polymer material such as rubber or any other suitable material or combination thereof that provides friction when pressed against a surface. In general, the friction element 106 is not restricted to a ring shape and may be any suitable device for providing an asymmetric bi-directional force in an autoinjector device 100. In some embodiments, the components of the autoinjector device 100 shown in FIGS. 1A and 1B are made of plastic, glass, a combination thereof, or any other suitable material commonly used in the medical device industry.

In some embodiments, the autoinjector device 100 is configured to provide haptic feedback. For example, the autoinjector device 100 may provide one or more of tactile and/or auditory feedback at any stage of delivery. As an example, a first haptic feedback may occur at the beginning of a transition from the storage to the launch position (i.e., beginning of injection), providing an indication to the user of the start of the transition. In an example, the autoinjector device 100 is a drug delivery device configured to deliver a drug to a user. In this case, the first haptic feedback occurs with the release of an actuator from one or more locking clips. The actuator may be a spring 220 and a locking clip may be a part of the puller 112, such that when the one or more locking clips are disengaged from the spring 220, the release provides the first haptic feedback.

In another example, the autoinjector device 100 is a demonstration device configured to simulate drug delivery. In this case, the first haptic feedback may be designed to simulate the first haptic feedback that occurs in a drug delivery device. The demonstration device may include a spring 220 that is weaker than the spring of the drug delivery device, such that feedback provided by the release of one or more locking clips on the puller 112 from the weaker spring 220 differs from the first haptic feedback provided by a drug delivery device. Thus, the demonstration device may include a separate mechanism for producing the first haptic feedback. The separate mechanism may include different components of the auotinjector device 100 moving with respect to one another. An example of a separate mechanism is described in more detail in relation to FIGS. 3A, 3B, and 3C.

FIGS. 3A, 3B, and 3C show cross sectional views of an autoinjector device 300 at different times during a transition from a storage position to a launch position. In particular, FIGS. 3A, 3B, and 3C illustrate how two haptic feedbacks may be generated. FIG. 3A shows a cross sectional view of the autoinjector device 300 in the storage position, similar to the storage position shown in FIG. 1A. FIG. 3B shows the autoinjector device 300 shortly after a transition from the storage position to the launch position is initiated, and FIG. 3C shows the autoinjector device 300 near a conclusion of the transition from the storage position to the launch position. The autoinjector device 300 is similar to the autoinjector device 100 shown in FIGS. 1A and 1B, with a few exceptions. In particular, the base element 108 of the autoinjector device 100 is replaced with a tube 334 and a base 336, where the tube 334 is configured to move within a restricted range of the base 336. Furthermore, the ridge 116 of the base element 108 in the autoinjector device 100 is replaced with a wider ridge portion 339 on the tube 334 of the autoinjector device 300. The wider ridge portion 339 is positioned within the base 336 and restricts the movement of the tube 334.

In FIG. 3A, the autoinjector device 300 is in a storage position. In particular, the notched region 104 is near a top portion of the tube 334, and the wider ridge portion 339 is near a top portion of the base 336, such that a small gap exists at a region 335 of the base 336, and no gap exists at a region 337 of the base 336. When the transition from the storage position of FIG. 3A to the launch position begins, the plunger 102 moves in the direction 109, and the friction element 106 about the notched region 104 of the plunger 102 contacts an inner wall 332 of the tube 332. A magnified view of the friction element 106 about the notched region 104 during the transition from the storage position to the launch position is shown in FIGS. 4A and 4B. The frictional contact between the friction element 106 and the inner wall 332 urge the tube 334 to move in the direction 109, until a bottom of the tube 334 contacts a portion of an end cap 338, as shown in FIG. 3B.

FIG. 3B shows the autoinjector device 300 shortly after the transition from the storage position to the launch position has initiated. In particular, during the transition between the storage position shown in FIG. 3A and the launch position shown in FIG. 3C, the spring 320 urges the plunger 102, the puller 112, and the piston 105 in the direction 109. Additionally, during the brief time between the storage position shown in FIG. 3A and the position shown in FIG. 3B, the tube 334 is also urged in the direction 109, until the tube 334 contacts the end cap 338. As shown in FIG. 3B, the tube 334 has moved from its position in FIG. 3A in the direction 109 such that the region 337 includes a small gap, and the region 335 does not include a gap. The contact between the tube 334 and the end cap 338 occurs near the region 335 and gives rise to a first haptic feedback, signaling to the user that the transition has begun or that discharge has begun is about to begin. In particular, a bottom surface of the wider ridge portion 339 contacts a top surface of the end cap 338. In some implementations, both the bottom surface of the wider ridge portion 339 and a top surface of the end cap 338 are substantially circular, such that the contact occurs around a circumference of the tube 334 and the end cap 338. In other implementations, the wider ridge portion 339 includes discrete portions which contact the end cap 338, or a top surface of the end cap 338 includes discrete portions that contact the wider ridge portion 339. In either case, the contact provides a first haptic feedback indicating that the transition from the storage position to the launch position has begun. After the first haptic feedback is provided, the plunger 102 and the puller 112 continue to travel in the direction 109 until the transition to the launch position is complete.

FIG. 3C shows the autoinjector device 300 at an end of the transition from the storage position to the launch position (i.e., end of injection). In particular, a second haptic feedback is provided at the end of the transition, signaling to the user that the transition is complete or that discharge is complete. At the conclusion of the transition from the storage position to the launch position, the notched region 104 travels in the direction 109 and passes the bottom portion of the tube 334, near the region 335. When the friction element 106 passes through the bottom portion of the tube 334, the friction element 106 reaches the widened base 336 and is released from being pressed against the inner wall 332 of the tube 334. Because the spring 320 continues to urge the plunger 102 in the direction 109, the release of the friction element 106 causes a sudden acceleration in the movement of the plunger 102 in the direction 109. The sudden acceleration lasts for a short time period because the lip 119 at the base of the puller 112 (which is fixedly attached to the plunger 102) contacts an element 121 of the housing 101. In another example, the lip 114 of the piston 105 may contact a top surface of the base 336. As described in relation to FIG. 3B, the contact between the lip 119 and the element 121 and/or between the lip 114 and the base 336 may occur substantially around a circumference of the surfaces of the components, or at discrete points. In either case, the contact causes the movement of the plunger 102 in the direction 109 to stop suddenly and provides a second haptic feedback. Thus, the release of the friction element 106 from being pressed against the inner wall 332 of the tube 334, and the resulting contact between the lip 119 and the element 121 and/or between the lip 114 and the base 336, causes the second haptic feedback signaling the end of the transition from the storage position to the launch position.

As shown in FIGS. 1A, 1B, 2, and 3A-3C the outer wall of the lip portion 114 is wider than the remainder of the puller 112, while the inner wall diameter of the lip portion 114 is the same as the remainder of the puller 112. By having a smooth inner wall surface, the base element 108 allows for smooth transitions between the launch and storage positions. When the plunger 102 enters the launch position shown in FIGS. 1B and 3C, contact may occur between the lip 114 and the element 121 and/or between the lip 119 and the ridge 116 (as shown in FIG. 1B) or between the lip 119 and the base 336 (as shown in FIG. 3C), causing the second haptic feedback. By producing one or more haptic feedbacks, the autoinjector device 100 provides a realistic user experience.

In some embodiments, the first and/or second haptic feedbacks in a demonstration device are calibrated to mimic the haptic feedbacks provided in a drug delivery device. For example, characteristics of a haptic feedback in a demonstration device may be compared to the same characteristics of a drug delivery device, and the demonstration device may be adjusted to match the characteristics of the drug delivery device. In an example, the sound level of the haptic feedbacks in a drug delivery device may be within a range 55 to 70 dB, and the demonstration device may be altered such that the haptic feedbacks of the demonstration device are within the same range. For example, to increase or decrease the loudness of a haptic feedback, one or more components of the device may be elongated or shortened with respect to one another. In particular, to change a sound level of the first haptic feedback, the height of the wider ridge portion 339 may be decreased relative to a height of the base 336, such that the tube 334 travels a longer distance before the contact at the region 335 occurs. Depending on a strength of the spring 320, this may result in a louder or a softer first haptic feedback. Similarly, to change a sound level of the second haptic feedback, the plunger 102 may be allowed to travel a longer distance before contact occurs between the lip 119 and the element 121 and/or between the lip 114 and the base 336. Depending on the strength of the spring 320, this may result in a louder or a softer second haptic feedback. In general, any suitable characteristic may be used, and any suitable targeted range of values may be used.

FIGS. 4A, 4B, 5A, and 5B show various views of an illustrative friction element 106 interacting with the inner wall of the base element 108. Referring now to FIGS. 4A and 4B and FIGS. 5A and 5B, each view includes a portion of the plunger 102 including the notched region 104 and a portion of the base element 108. The friction element 106 surrounds a portion of the notched region 104, which is depicted as having three regions including a proximal region 430, a central region 432, and a distal region 434. As shown, the regions 430, 432, and 434 have roughly the same height and different respective cross sections. In particular, the cross sections of the proximal region 430 and central region 432 are circular and have variable tapered diameters, such that the diameter is narrower at the central region 432 and is wider at the proximal region 430. The distal region 434 is asymmetrically shaped and includes a ridge 420, which includes a raised portion of the distal region 434. In particular, the ridge 420 extends across the width of the plunger 102 and is defined by two raised portions 436a and 436b and two lowered portions 438a and 438b in the distal region 434. The two raised portions 436a and 436b are on opposite sides of the distal region 434, and the two lowered portions 438a and 438b are also on opposite sides of the distal region 434.

Referring now to FIGS. 4A and 4B, these two views depict the notched region 104 during a transition of the autoinjector device 100 from the storage position (FIG. 1A) to the launch position (FIG. 1B). During this transition, the plunger 102 moves in the launch direction (downward arrow 431). Because the outer diameter of the friction element 106 is greater than the inner diameter D of the base element 108, the outer portion 433 of the friction element 106 presses against the inner wall 125 of the base element 108. The contact between the friction element 106 and the base element 108 produces a frictional force that resists the movement of the plunger towards the launch position. The frictional force urges the friction element 106 towards the proximal region 430 of the notched region 104. In addition, the inner portion of the friction element 106 may be sized appropriately to press against the outer wall of the notched region 104. As the friction element 106 is urged towards the proximal region 430 (i.e., the direction opposite of the arrow 431 in FIGS. 4A and 4B), the diameter of the notched region 104 increases. The increased diameter of the notched region 104 presses against the inner portion of the friction element 106. This causes the friction element 106 to press further against the inner wall of the base element 108, resulting in an increased frictional force. Thus, as the plunger 102 moves in the launch direction (i.e., the direction 431 in FIGS. 4A and 4B), the friction element 106 is urged towards the proximal region 430 and resists the movement of the plunger 102.

As shown in FIGS. 4A and 4B, the friction element 106 reaches the proximal region 430 (i.e., the portion of the notched region 104 with the widest diameter) during the transition of the autoinjector device 100 from the storage position to the launch position. The friction element 106 may reach this position before the plunger 102 reaches the launch position. In such cases, during the remainder of the transition to the launch position, the friction element 106 remains at this position relative to the notched region 104. In particular, the inner diameter of the friction element 106 may be relatively less than the diameter A of the body 113 of the plunger 102, such that the friction element 106 remains within the notched region 104 during the transition. When the friction element 106 is at the proximal region 430 (as shown in FIGS. 4A and 4B), the variable diameter B of the notched region 104 is wider than at the central region 432. In this case, the wider diameter of the proximal region 430 causes the friction element 106 to press against the inner wall of the base element 108.

After the autoinjector device 100 reaches the launch position (FIG. 1B), the proximal region 430 reaches the widened base portion 118 of the base element 108. The outer diameter of the friction element 106 may be sized appropriately to be larger than the diameter D of the tube portion 117 of the base element 108 but less than the diameter E of the widened base portion 118. In this case, when the friction element 106 approaches the junction between the tube portion 117 and the widened base portion 118, the friction element 106 may drop to the distal region 434 of the notched region 104. In an example, the actuator (such as the spring 220, the spring 320, a post, or any other suitable element for urging a movement of the plunger 102) urges the plunger 102 in the direction 109, such that when the friction element 106 reaches the widened base portion 118, the sudden release of the friction element 106 causes the friction element to move away from the proximal region 430 and towards the distal region 434 of the notched region 104. As described in relation to FIG. 3C, the sudden release of the friction element 106 also causes a sudden acceleration in the movement of the plunger 102 in the direction 109. The sudden acceleration lasts for a short time period, because one or more components fixedly attached to the plunger 102 may contact one or more other components, causing the movement of the plunger 102 in the direction 109 to stop suddenly and providing a haptic feedback.

Referring now to FIGS. 5A and 5B, these two views include the notched region 104 during a transition of the autoinjector device 100 from the launch position (i.e., FIG. 1B) to the storage position (i.e., FIG. 1A). During this transition, an actuator (such as the spring 220, the spring 320, a post, or any other suitable element for urging a movement of the plunger 102) urges the plunger 102 in the direction 541, and the friction element 106 bends around the ridge 420. In particular, the bending results in two raised portions 540a and 540b (generally raised portion 540) and two lowered portions 542a and 542b (generally lowered portion 542) of the friction element 106. During the movement of the plunger 102 in the storage direction, the lowered portion 542 of the friction element 106 presses against the inner wall of the base element 108, as shown in FIG. 5B. Thus, while the plunger 102 moves towards the storage position, the lowered portion 542 is urged towards the lowered portion of the distal region 434. In addition, the raised portion 540 of the friction element 106 is positioned at the raised portion of the distal region 434. As shown in FIG. 5A, because the friction element 106 is bent, the raised portions 540a and 540b do not press against the inner wall of the base element 108, resulting in a decreased amount of frictional force to resist the movement of the plunger 102. Therefore, during the transition of the autoinjector device 100 from the launch position to the storage position, the ridge 420 causes the friction element 106 to exert less frictional force during the transition to the storage position compared to the transition to the launch position.

The configuration of the friction element 106 and the notched region 104 as shown in FIGS. 4A, 4B, 5A, and 5B is an example of a system for providing bi-directional forces. In particular, while the autoinjector device 100 transitions from the storage to launch positions, the friction element 106 and the notched region 104 provide a frictional force resisting the movement of the plunger 102. During a return from the launch position to the storage position, the ridge 420 of the notched region 104 causes the friction element 106 to provide a reduced amount of frictional force resisting the movement of the plunger 102. A reduced resistive force while the autoinjector device 100 returns to the storage position may be desirable for conveniently resetting the autoinjector device 100. For example, it may be desirable to use the device multiple times for demonstration purposes. By reducing the resistive force during the return of the device to the storage position, the resetting of the device occurs efficiently and is user friendly by allowing the user to exert less force to recharge the device.

As shown in FIGS. 4A, 4B, 5A, and 5B, the ridge 420 has two raised portions on opposite sides of the notched region 104. This is shown for illustrative purposes only and one of ordinary skill in the art will understand that, in general, any number of raised portions may be used if it is desirable to have different amounts of resistive forces during the upward movement of the plunger 102. In addition, as shown in FIGS. 4A, 4B, 5A, and 5B, the proximal region 430, central region 432, and distal region 434 have similar heights. In general, the proportions of the regions 430-434 may be varied depending on the desired dimensions of the other components of the autoinjector device 100.

As described herein, the interaction between the friction element 106 and different portions of the notched region 104 causes asymmetric bi-directional forces to be exerted during transitions of the autoinjector device 100 between storage and launch positions. This mechanism can be used in place of, or in addition to, using other mechanisms of an autoinjector device.

FIG. 6 shows an illustrative flow diagram of a method 600 for applying asymmetric bi-directional forces while an autoinjector device 100 transitions between storage and launch positions. The method 600 includes the steps of applying a first amount of resistance against movement of a plunger 102 from a storage position to a launch position (step 602), allowing the plunger 102 to return to the storage position (step 604), and applying a second amount of resistance against the movement of the plunger 102 from the launch position to the storage position, wherein the second amount of resistance is less than the first amount (step 606).

At step 602, a first amount of resistance is applied against a movement of the plunger 102 from a storage position to a launch position. In particular, the storage position of the autoinjector device 100 may be as depicted in FIG. 1A, and the launch position of the autoinjector device 100 may be as depicted in FIG. 1B. While the plunger 102 moves from the storage position to the launch position (i.e., in the direction of the arrow 109 in FIG. 1A), the first amount of resistance is applied by the frictional force between the friction element 106 and the inner wall of the tube portion 117 of the base element 108. In particular, the frictional force provides a first amount of resistance against the movement of the plunger 102.

At step 604, the plunger 102 is allowed to return to the storage position, and at step 606, a second amount of resistance is applied against the movement of the plunger 102 towards the storage position. The second amount of resistance is less than the first amount of resistance applied at step 602. The steps 604 and 606 are performed simultaneously, such that the second amount of resistance is applied while the plunger 102 returns to the storage position. As described in relation to FIGS. 5A and 5B, the second amount of resistance is reduced compared to the first amount because of the ridge 420 in the notched region 104. The ridge 420 causes the friction element 106 to bend, thereby pressing less against the inner wall of the base element 108 and exerting less frictional force. The reduced amount of frictional force results in a smaller amount of resistance against the movement of the plunger 102 in the storage direction (i.e., arrow 111 in FIG. 1B).

FIG. 7 shows an illustrative flow diagram of a method 700 for operating an autoinjector device 100. The method 700 includes the steps of a plunger 102 resting in a storage position (step 702), initiating a movement of the plunger 102 from a storage position to the launch position (step 704), and providing a first haptic feedback (step 706). The method 700 further includes continuing movement of the plunger 102 from the storage position to the launch position (step 708), the plunger 102 entering the launch position (step 710), and providing a second haptic feedback (step 712). The first and/or second haptic feedbacks may include one or more tactile and/or auditory feedback during delivery.

At step 702, the plunger 102 is in the storage position (i.e., as shown in FIG. 1A). At step 704, a user initiates a movement of the plunger 102 from the storage position to the launch position. In particular, the transition of the plunger 102 from the storage position to the launch position may be referred to a discharge of the autoinjector device 100. In order to initiate the discharge, the user may press a button on the autoinjector device 100, or may press one end of the autoinjector device 100 onto an area to receive medication. When the autoinjector device 100 is used for demonstration purposes, the user may press the autoinjector device 100 onto an area of skin, a mannequin, or any other suitable surface for demonstration the use of an autoinjector device.

At step 706, the autoinjector device 100 provides a first haptic feedback after the discharge of the autoinjector device 100 is initiated. In particular, the first haptic feedback may be provided soon after the user initiates the discharge. The first haptic feedback may be provided by releasing an actuator from at least one locking clip. In particular, the actuator may be a spring 220 or 320 and a locking clip may be a part of the puller 112 or the piston 105. In addition or alternatively, the first haptic feedback may be provided when contact between two components of the autoinjector device 100 or 300 contact, such as was described in detail in relation to FIG. 3B. The first haptic feedback is desirable in a demonstration device for emulating the feedback that may result when initiating discharge of a drug delivery device. In particular, a drug delivery device may use the first haptic feedback to indicate to the user that discharge is beginning. Even though the user initiates the movement at step 704 (by pressing a button or pressing the device against a surface, for example), the first haptic feedback provides confirmation to the user that the movement has initiated. In a drug delivery autoinjector device 100, the first haptic feedback indicates to the user that the needle is about to be inserted. Because a demonstration device should emulate a user's experience with a drug delivery device, it is desirable to include the first haptic feedback in the demonstration device as well as in the drug delivery device.

At step 708, the movement of the plunger 102 of the autoinjector device 100 from the storage position (e.g., as shown in FIG. 1A) to the launch position (e.g., as shown in FIG. 1B) continues, and at step 710, the plunger 102 enters the launch position. At step 712, the autoinjector device 100 provides a second haptic feedback when the plunger enters the launch position. As was described in relation to FIG. 3C, the second haptic feedback may result from contact between the lip 119 at the base of the puller 112 and an element 121 on the housing 101. The second haptic feedback may also or alternatively result from contact between the lip 114 of the piston 105 and the base 336 of FIG. 3C. The second haptic feedback may be used in a drug delivery device to indicate to the user that the discharge is complete. Completion of the discharge may include completion of insertion of the needle in a drug delivery device, completion of medication delivery, completed retraction of the needle, any other suitable indication of an autoinjector device, or any combination thereof. Because a demonstration device should emulate a user's experience with a drug delivery device, it is desirable to include the first haptic feedback in the demonstration device as well as in the drug delivery device.

As described herein, the autoinjector device 100 is configured to provide two haptic feedbacks: a first haptic feedback to indicate initiation of discharge of the autoinjector device 100 and a second haptic feedback to indicate completion of the discharge. In general, the autoinjector device 100 may provide any number of haptic feedbacks for indicating to the user any of the various stages of the injection process.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. An autoinjector device, comprising:
a housing;
a plunger slidably mounted to the housing, the plunger including a tapered region;
an actuator for urging the plunger with respect to the housing from a storage position to a launch position; and
a friction element about a portion of the tapered region for resisting a movement of the plunger from the storage position to the launch position.

2. The autoinjector device of claim 1, wherein the tapered region has a variable diameter.

3. The autoinjector device of claim 2, wherein:
the tapered region has a central region between a proximal region and a distal region;
the proximal region is closer to the storage position and the distal region is closer to the launch position.

4. The autoinjector device of claim 3, wherein the proximal region has a diameter larger than a diameter of the central region.

5. The autoinjector device of claim 3, wherein the distal region has a diameter larger than a diameter of the central region.

6. The autoinjector device of claim 3, wherein the tapered region is tapered between the distal and central regions and between the proximal and central regions.

7. The autoinjector device of claim 1, wherein the friction element presses against a wall of the housing when the plunger is urged from storage position to the launch position, resulting in a frictional resistive force.

8. The autoinjector device of claim 1, wherein the movement of the plunger from the storage position to the launch position causes a position of the friction element to be at a proximal region of the tapered region.

9. The autoinjector device of claim 1, wherein the plunger is configured to return to the storage position from the launch position.

10. The autoinjector device of claim 9, wherein the tapered region includes a mechanism for reducing a resistive effect of the friction element when the plunger returns to the storage position from the launch position relative to a resistive effect of the friction element when the plunger moves from the storage position to the launch position.

11. The autoinjector device of claim 10, wherein the mechanism causes the friction element to bend when the plunger returns to the storage position from the launch position, wherein bending of the friction element results in a reduced amount of friction between the friction element and a wall of the housing.

12. The autoinjector device of claim 10, wherein the mechanism is a ridge on the distal region of the tapered region.

13. The autoinjector device of claim 1, wherein the autoinjector device is a demonstration device used for simulating auto-injection.

14. The autoinjector device of claim 1, wherein the autoinjector device is configured to provide a first haptic feedback at an initiation of the plunger's movement from the storage position to the launch position.

15. The autoinjector device of claim 14, wherein a release of the actuator from at least one locking clip provides the first haptic feedback.

16. The autoinjector device of claim 14, further comprising a puller coupled to the plunger and a cap, wherein the puller and the cap are configured to contact, thereby providing the first haptic feedback.

17. The autoinjector device of claim 14, wherein the autoinjector device is configured to provide a second haptic feedback at a conclusion of the plunger's movement from the storage position to the launch position.

18. The autoinjector device of claim 17, wherein a release of the friction element causes the plunger to accelerate.

19. The autoinjector device of claim 18, wherein the second haptic feedback is provided when portions of the device contact.

20. The autoinjector device of claim 19, wherein the portions include a base and a puller coupled to the plunger.

21. The autoinjector device of claim 20, wherein the puller is configured to contact an outer wall of a widened portion of the base.

22. The autoinjector device of claim 1, wherein the autoinjector device is a medical device used for administering medication.

23. The autoinjector device of claim 22, wherein the plunger is a part of a syringe assembly including a needle and a fluid container.

24. The autoinjector device of claim 1, wherein the friction element is a rubber ring.

25. A method for actuating an autoinjector, the method comprising:
applying a first amount of resistance against a movement of a plunger slidably mounted to a housing from a storage position to a launch position; and
applying a second amount of resistance against a return of the plunger from the launch position to the storage position, wherein the second amount of resistance is smaller than the first amount of resistance.

26. The method of claim 25, wherein the resistance against the movement is caused by friction resulting from contact between a friction element about a portion of the plunger and a wall of the housing.

27. The method of claim 25, further comprising providing a first haptic feedback when the movement of the plunger from the storage position to the launch position initiates.

28. The method of claim 25, further comprising providing a second haptic feedback when the movement of the plunger from the storage position to the launch position concludes.

29-31. (canceled)
